(19) United States
(12) Patent Application Publication
(10) Pub. No.: US 2011/0230873 A1
(43) Pub. Date: Sep. 22, 2011

(54) Method mini refrigerator and apparatus for treating diseases

(76) Inventors: Mark Chak, Brooklyn, NY (US); Alexander Chak, Brooklyn, NY (US)

(21) Appl. No.: 12/661,630

(22) Filed: Mar. 22, 2010

(51) Int. Cl. A61B 18/02

(52) U.S. Cl. 606/21

(57) ABSTRACT

Treating diseases includes introducing into an unhealthy inclusion in an organ a needle and freezing the unhealthy inclusion, thereafter withdrawing the unhealthy inclusion deformed by freezing from the organs by the needle, and thereafter heating the area of the organ from which the unhealthy inclusion has been removed by the needle in the above specified sequence, which can be performed by individual, separate needles, or by a single needle.

### BACKGROUND OF THE INVENTION

The present invention relates to a method and apparatus for treating diseases for example, cancer, aids, polyps, prostate tumors, etc.

Methods and apparatuses for treatment of diseases including action on an unhealthy inclusion in an organ are known in great varieties. Some of them are disclosed in U.S. patent application publication 2004/0024392; U.S. Pat. No. 5,484,399; publication Hofmann, et al “Lowering of Tumor Interstetal Fluid Pressure Reduces Tumor Cell Proliferation in Xenograft Tumor Model” and my U.S. patent application Ser. No. 11/715,121. It is I now that the existing methods Mini refrigerator and apparatuses can be further improved. Also dis method good for animals.

### SUMMARY OF THE INVENTION

Accordingly, it is an object of the present invention to provide a method and an apparatus for treating diseases for example cancer tumors, aids, polyps, prostate tumors, etc, which is a further improvement of the existing methods and apparatuses.

In keeping with these objects and with others which will become apparent hereinafter, one feature of the present invention resides, briefly stated, in a method of treating diseases, comprising the steps of introducing into an unhealthy inclusion in an organ a needle and freezing the unhealthy inclusion, thereafter withdrawing the unhealthy inclusion deformed by freezing from the organs by the needle; and thereafter heating the area of the organ from which the unhealthy inclusion has been removed by a needle; and performing the above specified steps in the above specified sequence.

Another features of the present invention resides, briefly stated, in an apparatus for treating diseases, comprising a needle for introducing into an unhealthy inclusion in an organ a needle and freezing the unhealthy inclusion, a needle for thereafter withdrawing the unhealthy inclusion deformed by freezing from the organs; and a needle for thereafter heating the area of the organ from which the unhealthy inclusion has been removed; in the above specified sequence.

In accordance with a further advantageous feature of the present invention, the steps of the method can be performed with the use of a single needle, and the mini refrigerator, apparatus can use a single needle connectable to various means for providing successive freezing, withdrawal and heating as specified herein above.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### BRIEF DESCRIPTION OF THE DRAWINGS

FIG. 1 is a view showing a first step of the method in accordance with the present invention and the components of the inventive mini refrigerator used therefor;

FIG. 2 is a view showing a second step of the method in accordance with the present invention and the components of the inventive apparatus used therefor;

FIG. 3 is a view showing a third step of the method in accordance with the present invention and the components of the inventive apparatus used therefor; and

FIGS. 4 and 5 show another embodiment of the present invention with a component performing all three steps in a sequential order in accordance with the present invention.

### DESCRIPTION OF THE PREFERRED EMBODIMENTS

In accordance with a method for treating diseases performed in accordance with the present invention, a needle 1 is introduced into an organ with an unhealthy inclusion. A cooling medium (O-C), for example Freon is introduced from a cooling aggregate 2 by a compressor 3 through a recirculating pipe 4 and the needle 1 is cooled to a very low temperature. The temperature of the cooling medium is sensed by a sensor S and can be adjusted by thermostat T. The cooled needle which is introduced into the unhealthy inclusion freezes the unhealthy inclusion by subjecting it to a high level of cold.

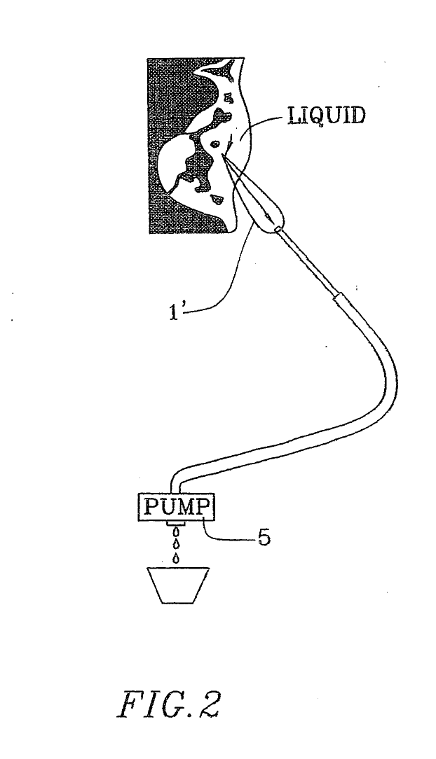

In a second step shown in FIG. 2, the unhealthy inclusion subjected to the high level of cold in the first step is withdrawn from the organ. A needle 1′ is introduced into the unhealthy inclusion and an area around it in the organ and its opposite end is connected with a pump 5. As a result, the substance of the unhealthy inclusion which was previously subjected to high cold and to a freeze it is withdrawn from the organ in which the unhealthy inclusion was incorporated before.

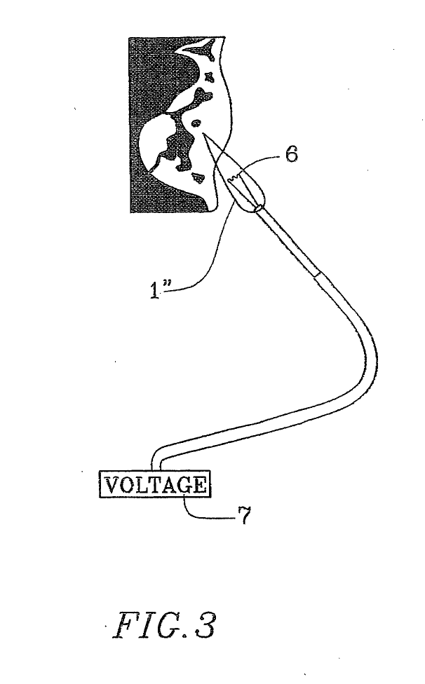

In a next step shown in FIG. 3, a needle 1″ provided with a heating device 6 is introduced in the area of the organ from which the unhealthy inclusion was removed. The heating device 6 is supplied with power from a source 7. The needle 1″ is heated and heats the area from which the unhealthy inclusion was removed.

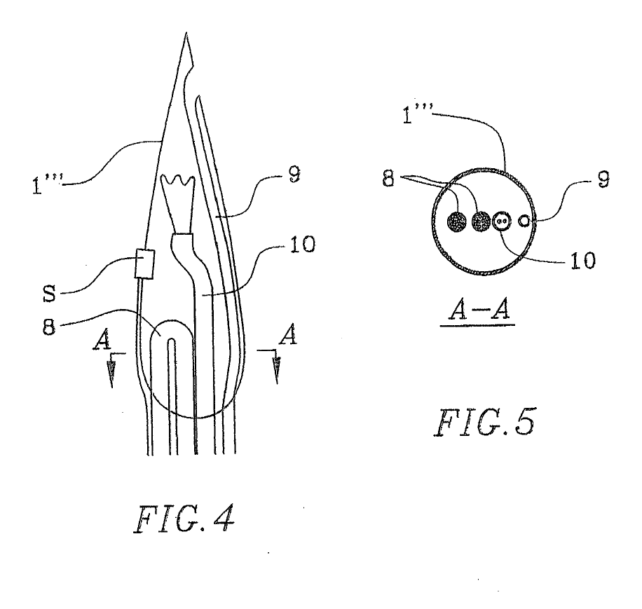

FIGS. 4 and 5 show a further embodiment of the present invention. Here the needle 1′″ has a cooling channel 8 for supplying liquid, and a withdrawing channel 9 for withdrawing, and a heating channel 10 with heating elements.

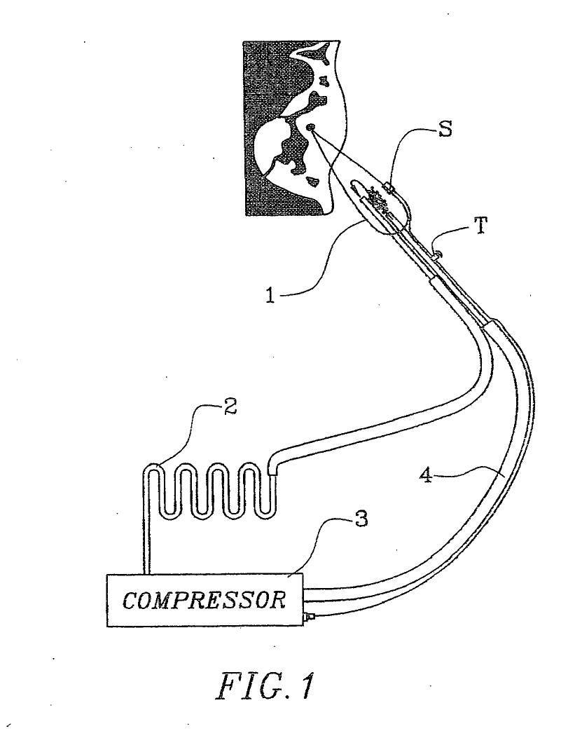

During a first step of the method in accordance with the present invention shown in FIGS. 4 and 5 the cooling channel 8 is connected to a cooling aggregate 2 with the compressor 3 through the pipe 4 shown in FIG. 1 so that the needle 1′″ is cooled and subjects the unhealthy inclusion to a high level of cold. In the second step the withdrawing channel 9 is connected to a pump, so that the unhealthy inclusion after freezing is withdrawn from the organ through the needle 1′″. In the third step heating element (for example a spiral) in the heating channel 10 is connected with the heat source 7 to provide heating of the heating device 6 and as a result of the needle so as to heat the area from which the unhealthy inclusion was withdrawn.

It will be understood that each of the elements described above, or two or more together, may also find a useful application in other types of methods and constructions differing from the type described above.

While the invention has been illustrated and described as embodied in method Mini refrigerator and apparatus for treating diseases, it is not intended to be limited to the details shown, since various modifications and structural changes may be made without departing in any way from the spirit of the present invention.

Without further analysis, the foregoing will so fully reveal the gist of the present invention that others can, be applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. A method of treating diseases, comprising the steps of introducing into an unhealthy inclusion in an organ a needle and freezing the unhealthy inclusion, thereafter withdrawing the unhealthy inclusion deformed by freezing from the organ by the needle; thereafter heating the area of the organ from which the unhealthy inclusion has been removed by the needle; performing the above specified steps in the above specified sequence; and using the single needle which provides consecutively the freezing of the unhealthy inclusion, the withdrawal of the unhealthy inclusion after the freezing, and the heating the area of the organ from which the unhealthy inclusion has been removed for drying the area, through separate cooling, withdrawing and heating channels provided in said single needle.

2-6. (canceled)

7. A method as defined in claim 1; and further comprising connecting to the separate cooling, withdrawing and heating channels of the single needle to a cooling aggregate, a pump, and a heating element to carry out the freezing, the withdrawal and the heating correspondingly.

8. A method as defined in claim 1; and further comprising heating the needle by air supplied through the heating channel so as to heat the needle and thereby heat the area of the organ from the unhealthy inclusion which has been removed by the needle for drying the area.
